(19) United States
(12) Patent Application Publication
(10) Pub. No.: US 2023/0043982 A1
(43) Pub. Date: Feb. 9, 2023

(54) ANTIPERSPIRANT COMPOSITIONS

(71) Applicants: The Procter & Gamble Company, Cincinnati, OH (US); University of Cincinnati, Cincinnati, OH (US)

(72) Inventors: Stevan David JONES, Hyde Park, OH (US); David Frederick SWAILE, Cincinnati, OH (US); Harshita Kumari, Mason, OH (US); Chandra A. ADE-BROWNE, Elmont, NY (US); Arnab DAWN, Cincinnati, OH (US)

(21) Appl. No.: 17/859,040

(22) Filed: Jul. 7, 2022

Related U.S. Application Data
(60) Provisional application No. 63/222,129, filed on Jul. 15, 2021.

(51) Int. Cl. A61K 8/29; A61Q 15/00; A61K 8/26; A61K 8/19; A61K 8/27; A61K 8/44; A61K 8/46; A61K 8/04

(52) U.S. Cl. CPC A61K 8/29; A61Q 15/00; A61K 8/26; A61K 8/19; A61K 8/27; A61K 8/44; A61K 8/46; A61K 8/042; A61K 8/046; A61K 2800/77; A61K 2800/874

(57) ABSTRACT

An aqueous antiperspirant composition comprising: 1) a metal salt with a M:X mole ratio from about 0.25 to 0.5, wherein X is an anion with a −1 or −2 charge and M is a metal ion with a +2, +3 or +4 charge; 2) an amino acid wherein the M:amino acid mole ratio is from about 5 to about 0.33; and 3) wherein the pH of the composition is from about 2 to about 4.

### FIELD OF THE INVENTION

The present disclosure relates to antiperspirant and deodorant compositions that contain metal salts and amino acids.

### BACKGROUND OF THE INVENTION

Most marketed antiperspirant actives are based on metal salts. These metal salts reduce sweating by forming a blockage at and within the duct entrance via hydration and neutralization of the salt by components of the sweat and skin. Historically, this process has been referred to as a precipitation of these salts within the sweat duct—i.e. a phase separation. However, recent observations highly suggest this hydration and neutralization process actually results in the formation of highly viscous metal hydroxide hydrogel complexes.

Many metal salt antiperspirant actives, such as aluminum chlorohydrate, are partially neutralized to improve skin compatibility versus un-neutralized salts such as aluminum chloride or iron chloride. Unfortunately, this neutralization can alter the pH and/or pH range on the skin wherein these actives can form these highly viscous hydrogels, resulting in a significantly reduced viscosity gel mass. As a result, these neutralized actives are often used at high concentrations, which can result in undesirable skin feel such as irritation, stickiness and visible white residue on skin, hair, and clothes.

Surprisingly, it has been discovered that adding amino acids to aqueous solutions of metal salts with small ionic radii and having +2, +3 or +4 charges can maintain or increase the pH range wherein highly viscous hydrogels are formed, and also maintain or increase the overall viscosity of the resulting hydrogel. As a result, we believe these combinations can provide high sweat control efficacy at low metal salt concentrations, thereby reducing or mitigating negative skin compatibility, skin feel and visible white residue negatives that we would expect from typical use of other antiperspirant actives.

### SUMMARY OF THE INVENTION

An aqueous antiperspirant composition comprising: 1) a metal salt with a M:X mole ratio from about 0.25 to 0.5, wherein X is an anion with a −1 or −2 charge and M is a metal ion with a +2, +3 or +4 charge; 2) an amino acid wherein the M:amino acid mole ratio is from about 5 to about 0.33; and 3) wherein the pH of the composition is from about 2 to about 4.

### BRIEF DESCRIPTION OF THE DRAWINGS

The figures herein are illustrative in nature and are not intended to be limiting.

FIG. 1 is a graph of viscosity vs. mole ratio of metal to amino acid for aluminum chloride: glycine.

FIGS. 2 and 3 are graphs that show the impact of amino type versus a tricarboxylic acid on hydrogel viscosity across a relevant range of pH values.

FIG. 4 is a graph of viscosity vs. pH for a 1:1 mole ratio of iron chloride: glycine.

### DETAILED DESCRIPTION OF THE INVENTION

As used herein, the articles “a” and “an” when used in a claim, are understood to mean one or more of what is claimed or described. As used herein, the terms “include,” “includes,” and “including” are meant to be non-limiting. The compositions of the present disclosure can comprise, consist essentially of, or consist of, the components of the present disclosure.

The terms “substantially free of or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or less than 0.1%, or less than 0.01%, or even 0%, by weight of the composition.

As used herein the phrase “antiperspirant and deodorant compositions” refers to compositions, including but not limited to, roll-ons, gels, clear gels, solid sticks, aerosols, and soft-solid sticks. For example, the antiperspirant or deodorant composition may be a composition such as a soft-solid deodorant, soft-solid antiperspirant, an invisible solid deodorant, an invisible solid antiperspirant, aerosol antiperspirant, fluid antiperspirant, body powder, or body spray. Such antiperspirant and deodorant compositions include compositions that are applied to at least a portion of the body, and which are used to combat body odor. “Soft solid” refers to a composition with a static yield stress of about 200 Pa to about 1,500 Pa after dispensing.

Unless otherwise noted, all component or composition levels are in reference to the active portion of that component or composition, and are exclusive of impurities, for example, residual solvents or by-products, which may be present in commercially available sources of such components or compositions.

All temperatures herein are in degrees Celsius (° C.) unless otherwise indicated. Unless otherwise specified, all measurements herein are conducted at 20° C. and under the atmospheric pressure.

In all embodiments of the present disclosure, all percentages are by weight of the total composition, unless specifically stated otherwise. All ratios are weight ratios, unless specifically stated otherwise.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Aqueous Antiperspirant Composition

The present invention includes an aqueous antiperspirant composition comprising:
1) a metal salt with a M:X mole ratio from about 0.25 to 0.5, wherein X is an anion with a −1or −2 charge and M is a metal ion with a +2, +3 or +4 charge;
2) an amino acid wherein the M:Amino Acid mole ratio if from about 5 to about 0.33; and
3) wherein the pH of the composition is from about 2 to about 4.

The aqueous antiperspirant compositions described herein may comprise a metal salt and an amino acid, wherein the pH of the composition is from about 2 to about 4. In some embodiments the pH may be from about 2 to about 5.

The metal salts of the present invention may comprise a metal that has a metal ion of +2, +3, or +4. Such metals may include, but are not limited to, aluminum, tin, zinc, gallium, iron, zirconium, titanium, magnesium, and hafnium. The metal salts may further comprise an anion with a −1 or −2 charge. Anions may include a chloride ion, but may alternatively be nitrate or sulfate ions. The metal salt may have a metal ion to anion mole ratio (M:X mole ratio) from about 0.25 to about 0.5. While not being bound by theory, it is believed that metal salts with such metal to anion mole ratios will form complexes at low pH value, rather than those salts which have higher ratios due to partial neutralization of the metal cation by a hydroxide ion.

The compositions may also comprise an amino acid, wherein the metal ion to amino acid mole ratio is from about 5 to about 0.33, preferably from about 0.5 to about 3. Suitable amino acids may include, but are not limited to, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. Some preferred amino acids may have an isoelectric point (pI) from about 5.5 to about 11, in some embodiments the isolectric point may be from about 5 (glycine) to about 11 (arginine). In some embodiments, glycine, cysteine, and arginine are preferred materials.

The composition comprises at most about 10%, in some embodiments at most about 20%, in some embodiments at most about 30%, by weight of the composition, of the anhydrous metal salt. This concentration measure excludes any waters of hydration from the determined weight percent. For example, if the metal salt is aluminum chloride hexahydrate (AlCl₃6H₂O), the six water molecules are not included in the calculation.

When using the Viscosity Test Method described herein, the inventive antiperspirant active may have a viscosity from 1,000 to 1,000,000 Pa·s. While not being bound by theory, it is believed that similar viscosity would be achieved by an antiperspirant composition comprising this active when used on the skin.

### Aqueous Antiperspirant Compositions

The present disclosure relates to antiperspirant compositions that may also in provide a deodorancy benefit. The inventive aqueous antiperspirant compositions described herein may be described as antiperspirant actives, and may be formulated into broader, commercially-available antiperspirant/deodorant compositions. Antiperspirant and deodorant compositions can be formulated in many forms. For example, antiperspirant and deodorant compositions can be, without limitation, a roll-on product, a stick including soft solid sticks and invisible solids, or an aerosol. Each of the aqueous antiperspirant compositions described below can further include a deodorant composition/active described herein and thus be described as an antiperspirant and/or deodorant composition. Each of the aqueous antiperspirant active composition or actives described herein may be included in any of the antiperspirant/deodorant forms.

### Water

The compositions of the present invention will comprise water, typically as a solvent for the metal salt and amino acid. Water can be present in an amount of about 1% to about 99.5%, about 25% to about 99.5%, about 50% to about 99.5%, about 75% to about 99.5% about 80% to about 99.5%, from about 15% to about 45%, or any combination of the end points and points encompassed within the ranges, by weight of the deodorant composition. In some embodiments, the composition may comprise at least about 5% water, or at least about 70% water.

### A. Roll-On and Gel

A roll-on or gel antiperspirant composition can comprise, for example, water, emollient, emulsifiers, deodorant, or combinations thereof in addition to the metal salts and amino acids of the present invention.

### Emollients

Roll-on or gel compositions can comprise an emollient system including at least one emollient but may also comprise a combination of emollients. Suitable emollients are often liquid under ambient conditions. Depending on the type of product form desired, concentrations of the emollient(s) in the deodorant or antiperspirant compositions can range from about 1% to about 95%, from about 5% to about 95%, from about 15% to about 75%, from about 1% to about 10%, from about 15% to about 45%, or from about 1% to about 30%, by weight of the deodorant or antiperspirant composition.

Emollients suitable for use in the roll-on and gel compositions include, but are not limited to, propylene glycol, polypropylene glycol (like dipropylene glycol, tripropylene glycol, etc.), diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, alkyl ethers of polyhydric and monohydric alcohols, volatile silicone emollients such as cyclopentasiloxane, nonvolatile silicone emollients such as dimethicone, mineral oils, polydecenes, petrolatum, and combinations thereof. One example of a suitable emollient comprises for roll-ons is PPG-15 stearyl ether. Examples of suitable emollients for a gel are cyclopentasiloxane and dimethicone. Other examples of suitable emollients or both forms include dipropylene glycol and propylene glycol.

### Emulsifier

The composition can contain an emulsifier. A suitable emulsifier can be, for example, a polymeric or nonpolymeric surfactants. Suitable surfactants are nonionic surfactants, cationic surfactants, amphoteric surfactants, zwitterionic surfactants, and mixtures thereof. Often roll-ons employ emulsifiers suitable for oil in water emulsions. Examples of emulsifiers for gel compositions include those suitable for water in silicone or water in oil emulsions.

Suitable emulsifiers for roll-on products include, for example, polyoxyethylene 2 stearyl ether, polyoxyethylene 20 stearyl ether, polyoxyethylene 21 stearyl ether, ceteth-10, ceteareth-2, and combinations thereof. Suitable emulsifiers for gel compositions include but are not limited to PEG/PPG-18/18 Dimethicone (DC5225), Lauryl PEG/PPG-18/18 methicone, Dimethicone/PEG-10/15 Crosspolymer, and combinations thereof.

When the emulsifier is present, it is typically present at a level of from about 0.01% to about 10%, alternatively from about 0.01% to about 3%, alternatively from about 0.05% to about 1%, alternatively from about 0.01% to about 0.05%, by weight of the composition.

### Deodorant Actives

Suitable deodorant actives can include any topical material that is known or otherwise effective in preventing or eliminating malodor associated with perspiration. Suitable deodorant actives may be selected from the group consisting of antimicrobial agents (e.g., bacteriocides, fungicides), malodor-absorbing material, and combinations thereof. For example, antimicrobial agents may comprise Piroctone olamine, cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

### B. Invisible Solid

Emulsified, invisible solid antiperspirant compositions as described herein can contain the metal salt and amino acid of the present invention, a primary structurant, emollients, emulsifiers a perfume, and additional chassis ingredient(s). The deodorant composition can further comprise other optional ingredient(s). The compositions can be in the form of a solid stick. The compositions can have a product hardness of about 600 gram force or more.

### Hardness

The invisible solid can have a product hardness of least about 600 gram. force, more specifically from about 600 gram. force to about 5,000 gram. force, still more specifically from about 750 gram. force to about 2,000 gram. force, and yet more specifically from about 800 gram. force to about 1,400 gram. force.

The term “product hardness” or “hardness” as used herein is a reflection of how much force is required to move a penetration cone a specified distance and at a controlled rate into an antiperspirant composition under the test conditions described herein below. Higher values represent harder product, and lower values represent softer product. These values are measured at 27° C., 15% relative humidity, using a TA-XT2 Texture Analyzer, available from Texture Technology Corp., Scarsdale, N.Y., U.S.A. The product hardness value as used herein represents the peak force required to move a standard 45-degree angle penetration cone through the composition for a distance of 10 mm at a speed of 2 mm/second. The standard cone is available from Texture Technology Corp., as part number TA-15, and has a total cone length of about 24.7 mm, angled cone length of about 18.3 mm, and a maximum diameter of the angled surface of the cone of about 15.5 mm. The cone is a smooth, stainless steel construction and weighs about 17.8 grams.

### Primary Structurants

The invisible solid can comprise a suitable concentration of a primary structurant to help provide the deodorant or antiperspirant with the desired viscosity, rheology, texture and/or product hardness, or to otherwise help suspend any dispersed solids or liquids within the composition.

The term “solid structurant” as used herein means any material known or otherwise effective in providing suspending, gelling, viscosifying, solidifying, and/or thickening properties to the composition or which otherwise provide structure to the final product form. These solid structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof.

The concentration and type of solid structurant selected for use in the deodorant and antiperspirant compositions will vary depending upon the desired product hardness, rheology, and/or other related product characteristics. For most structurants suitable for use herein, the total structurant concentration ranges from about 5% to about 35%, more typically from about 10% to about 30%, or from about 7% to about 20%, by weight of the composition.

Non-limiting examples of suitable primary structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fischer-Tropsch waxes, and microcrystalline wax; polyethylenes with molecular weight of 200 to 1000 daltons; solid triglycerides; behenyl alcohol, or combinations thereof.

Other non-limiting examples of primary structurants suitable for use herein are described in U.S. Pat. Nos. 5,976,514 and 5,891,424, the descriptions of which are incorporated herein by reference.

### Additional Chassis Ingredients

### Additional Structurant

The deodorant or antiperspirant composition can further comprise an additional structurant. The additional structurant may be present in an amount from 1% to about 10%, by weight of the composition. The additional structurant(s) will likely be present at an amount less than the primary structurant.

Non-limiting examples of suitable additional structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof.

Other non-limiting examples of additional structurants suitable for use herein are described in U.S. Pat. Nos. 5,976,514 and 5,891,424 .

### Emollient

The antiperspirant composition can comprise an emollient at concentrations ranging from about 20% to about 80%, and more specifically from about 30% to about 70%, by weight of the composition. The solvent can be a volatile silicone which may be cyclic or linear.

“Volatile silicone” as used herein refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976), which descriptions are incorporated herein by reference.

The volatile silicone can be a cyclic silicone having from 3 to 7, and more specifically from 5 to 6, silicon atoms, and still more specifically 5, like cyclopentasiloxane. These cyclic silicone materials will generally have viscosities of less than about 10 centistokes at 25° C. Linear volatile silicone materials suitable for use in the deodorant compositions include those represented by the formula:

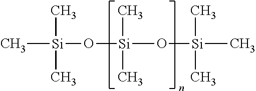

wherein n is from 1 to 7, and more specifically from 2 to 3. These linear silicone materials will generally have viscosities of less than about 5 centistokes at 25° C.

Specific examples of volatile silicone solvents suitable for use in the deodorant and antiperspirant compositions include, but are not limited to, Cyclomethicone D-5; GE 7207 and GE 7158 (commercially available from General Electric Co.); Dow Corning 344; Dow Corning 345; Dow Corning 200; and DC1184 (commercially available from Dow Corning Corp.); and SWS-03314 (commercially available from SWS Silicones).

### Non-Volatile Organic Emollients

Non-volatile organic fluids may be present, for example, in an amount of about 15% or less, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv.TM.), octyldodecanol, isostearyl isostearate, octododecyl benzoate, isostearyl lactate, isostearyl palmitate, and isobutyl stearate.

Suitable emulsifiers for emulsified stick products include, for example, polyoxyethylene 2 stearyl ether, polyoxyethylene 20 stearyl ether, polyoxyethylene 21 stearyl ether, ceteth-10, ceteareth-2, PEG/PPG-18/18 Dimethicone (DC5225), L airy′ PEG/PPG-18/18 methicone, Dimethicone/PEG-10/15 Crosspolymer, and combinations thereof

### C. Aerosols

Antiperspirant sprays of the present invention can comprise both single phase liquid and emulsions. Both forms may contain a propellant and a concentrate. The product concentrate may contain, for example water, additional carrier liquids, emollients, emulsifiers and a perfume.

### Propellant

The aerosol compositions described herein can include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3 -heptafluoropropane, trans-1,3,3,3 -tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 diflouroethane). The propellant may have a concentration from about 15%, 25%, 30%, 32%, 34%, 35%, 36%, 38%, 40%, or 42% to about 90%, 85% 80% 70%, 65%, 60%, 54%, 52%, 50%, 48%, 46%, 44%, or 42%, or any combination thereof, by weight of the total fill of materials stored within the container.

### Carrier

A carrier liquid suitable for use in a single phase liquid spray can include, water, alcohol, glycols, any other nonionc water soluble liquid or combinations thereof. The carrier may be present in an amount of about 1% to about 99.5%, about 25% to about 99.5%, about 50% to about 99.5%, about 75% to about 99.5% about 80% to about 99.5%, from about 15% to about 45%, or any combination of the end points and points encompassed within the ranges, by weight of the composition. A suitable example of an alcohol can include ethanol. Suitable examples of an glycol can include but are not limited to propylene glycol, propane diol, dipropylene glycol, tripropylene glycol and polyethylene glycols such as PEG 400.

Carrier liquids suitable for emulsion concentrates can include an oil or a mixture of two or more oils. The carrier may be present in an amount of about 1% to about 99.5%, about 25% to about 99.5%, about 50% to about 99.5%, about 75% to about 99.5% about 80% to about 99.5%, from about 15% to about 45%, or any combination of the end points and points encompassed within the ranges, by weight of the composition. Useful oils include, for example, volatile silicone oils and non-volatile organic oils. “Volatile silicone”, as used herein, refers to those silicone materials that have measurable vapor pressure under ambient conditions. Non-limiting examples of suitable volatile silicones are described in Todd et al., “Volatile Silicone Fluids for Cosmetics”, Cosmetics and Toiletries, 91:27-32 (1976). The volatile silicone can be a cyclic silicone having from at least about 3 silicone atoms or from at least about 5 silicone atoms but no more than about 7 silicone atoms or no more than about 6 silicone atoms. For example, volatile silicones can be used which conform to the formula:

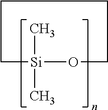

wherein n is from about 3 or from about 5 but no more than about 7 or no more than about 6. These volatile cyclic silicones generally have a viscosity of less than about 10 centistokes at 25° C. Suitable volatile silicones for use herein include, but are not limited to, Cyclomethicone D5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.). SWS-03314, SWS-03400, F-222, F-223, F-250, F-251 (available from SWS Silicones Corp.); Volatile Silicones 7158, 7207, 7349 (available from Union Carbide); MASIL SF-V (available from Mazer) and combinations thereof. Suitable volatile silicone oils can also include linear silicone oils such as, for example, DC200 (1 cSt), DC200 (0.65 cSt), and DC2-1184, all of which are available from Dow Corning Corp. In certain examples, the volatile silicone oil can have a viscosity of less than 10 centistokes at 25° C.

Non-volatile organic, emollient oils can also be employed. A representative, non-limiting list of emollient oils includes CETIOL CC (dicaprylyl carbonate), CETIOL OE (dicaprylyl ether), CETIOL S (diethylhexylcyclohexane), and CETIOL B (dibutyl adipate), all of which are available from Cognis, and LEXFEEL 7 (neopentyl glycol diheptanoate) from Inolex. In certain examples, the organic emollient oils have a viscosity of less than 50 centistokes at 25° C. The term “organic emollient oil” as used herein means silicon-free emollient oils that are liquid at 25° C., and that are safe and light to skin and can be miscible with volatile silicone oils (as described above).

The emulsion concentrate can contain an emulsifier. A suitable emulsifier can be, for example, a polymeric or nonpolymeric surfactants. Suitable surfactants are nonionic surfactants, cationic surfactants, amphoteric surfactants, zwitterionic surfactants, and mixtures thereof. Often roll-ons employ emulsifiers suitable for oil in water emulsions. Examples of emulsifiers for gel compositions include those suitable for water in silicone or water in oil emulsions.

Suitable emulsifiers for roll-on products include, for example, polyoxyethylene 2 stearyl ether, polyoxyethylene 20 stearyl ether, polyoxyethylene 21 stearyl ether, ceteth-10, ceteareth-2, and combinations thereof. Suitable emulsifiers for gel compositions include but are not limited to PEG/PPG-18118 Dimethicone (DC5225), Lauryl PEG/PPG-18/18 methicone, Dimethicon/PEG-10/15 Csosspolymer, and combinations thereof.

When the emulsifier is present, it is typically present at a level of from about 0.01% to about 10%, alternatively from about 0.01% to about 3%, alternatively from about 0.05% to about 1%, alternatively from about 0.01% to about 0.05%, by weight of the composition.

### Other Optional Ingredients

The compositions can further comprise any optional material that is known for use in antiperspirant and deodorant compositions or other personal care products, or which is otherwise suitable for topical application to human skin.

### Method of Using

The antiperspirant and deodorant compositions of the present invention may be topically applied to the axilla or other area of the skin in any known or otherwise effective method for controlling malodor associated with perspiration. These methods comprise applying to the axilla or other area of the human skin an effective amount of the deodorant composition of the present invention, typically about 0.1 gram per axilla to about 2.0 gram per axilla.

### TEST METHODS

It is understood that the test methods that are disclosed in the Test Methods Section of the present application should be used to determine the respective values of the parameters of Applicant's claimed subject matter as claimed and described herein.

### Viscosity Test Method:

Sample preparation for viscosity measurements:
10% w/v, the metal salt in water, was pH adjusted to target pH using 15% ammonium hydroxide aqueous solution added in 0.05 ml installments, under constant stirring, followed by vortexing for 10 S.
pH adjusted samples was then allowed to equilibrate for 20 hours at room temperature (−23° C.) before conducting viscosity measurements.
Viscosity measurements were run on Discovery HR-2 rheometer. A 40 mm cone-plate steel geometry with 55 mm truncation was used for all experiments. A flow-ramp experiment was performed after transferring about 2 ml of sample to the rheometer plate, kept at 25° C. Sample conditioning was done using a programmed soak time of 10 sec followed by equilibration time of 90 sec. The flow ramp step was performed at 25° C., using logarithmic mode for a duration of 240 sec. The shear rate was varied from 5×10⁻⁴s⁻¹ to 30 s⁻¹, with 100 data points per decade. The viscosity values for a sample was determined by measuring the viscosity value from the viscosity vs shear rate plot at minimum shear rate, using the software TRIOS.

### Examples

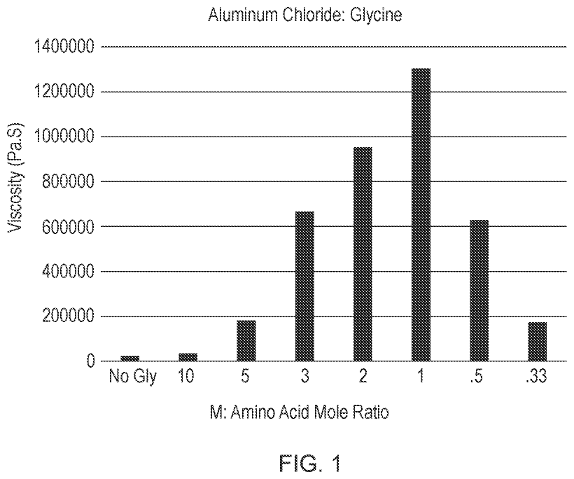

Viscosity test results for series of 10% samples of aluminum chloride with variable level of glycine are shown in FIG. 1. This data shows that viscosity has a maximum value near a 1:1 ratio but that viscosity increases from ratios less than about 5 and more than about 0.33. Whilst not being bound by theory, it is believed that this combination results in a hydrogel that will form more resilient blockages in or at the entrance to sweat duct.

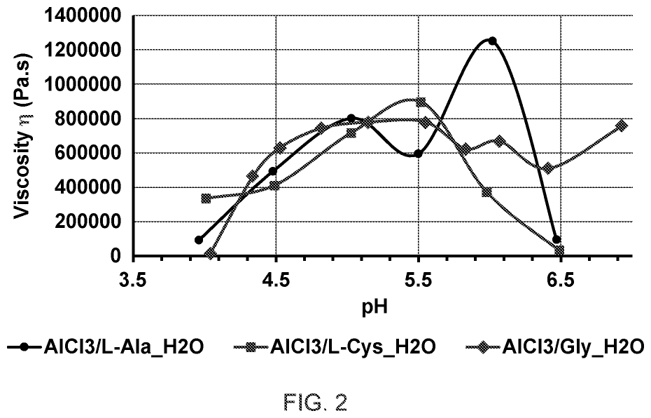

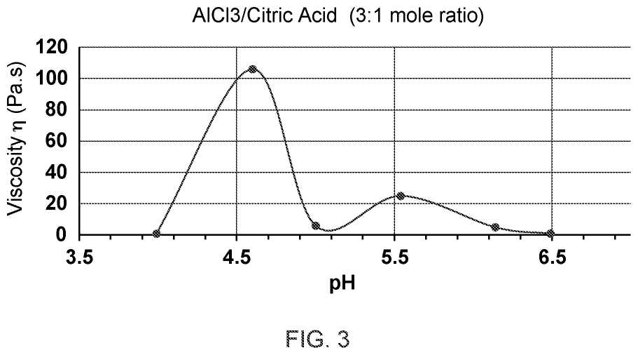

FIGS. 2 and 3 show the impact of amino type versus a tricarboxylic acid on hydrogel viscosity across a relevant range of pH values. As can be seen from the in FIG. 2, glycine, alanine, and cysteine at a 1:1 mole ratio or AlCl3 to amino acid, provide a high viscosity across the pH region. Conversely in FIG. 3, citric acid does not provide a similar viscosity benefit and as such would not be expected to provide the desired efficacy benefit. While not being bound by theory, it is believed that the amine function group will be protonated in this relevant pH region and as such will alter the hydrogel structure and result in the desired viscosity increase.

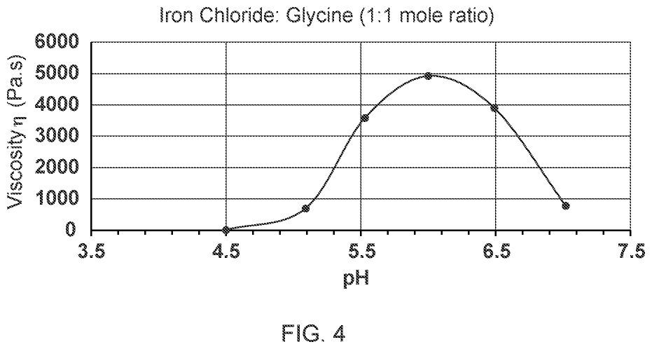

FIG. 4 shows data from the same methodology as FIG. 2 but use iron chloride (FeCl₃) in combination with glycine at a M:amino acid mole ratio of 1. While iron does not provide the level of viscosity seen with aluminum, it is believed that this level of viscosity will provide a sweat reduction benefit when applied to axilla.

Non Limiting examples of antiperspirant active compositions:

| | Example | Example | Example | Example | Example |
|---|---|---|---|---|---|
| | A | B | C | D | E |
| Aluminum | 3.0 | 6.0 | 10.0 | | |
| Chloride | | | | | |
| (AlCl₃) | | | | | |
| Iron | | | | 5.0 | 9.0 |
| Chloride | | | | | |
| (FeCl₂) | | | | | |
| Glycine | 1.7 | | | 6.0 | 1.8 |
| Cysteine | | 16.4 | | | |
| Arginine | | | 2.6 | | |
| Water | 95.3 | 77.6 | 87.4 | 89.0 | 89.2 |
| M:X | 0.33 | 0.33 | 0.33 | 0.33 | 0.33 |
| mole ratio | | | | | |
| M:AA | 1.0 | 0.3 | 5.0 | 0.5 | 3.0 |
| mole ratio | | | | | |

| | Example | Example |
|---|---|---|
| | F | G |
| Titanium | 5 | |
| Chloride | | |
| (TiCl3) | | |
| Zinc | | 7.5 |
| Chloride | | |
| (ZnCl2) | | |
| Glycine | 4.8 | |
| Cysteine | | 20 |
| Water | | 72.5 |
| M:X | 0.33 | 05. |
| mole ratio | | |
| M:AA | 0.5 | 0.33 |
| mole ratio | | |

The following are examples of antiperspirant and deodorant compositions comprising the antiperspirant active composition.

| | Emulsion stick | Gel | Roll-on | Spray |
|---|---|---|---|---|
| Cyclopentasiloxane | 21.3 | 7.0 | | 3.2 |
| Dow 5200 formulation aid | 1.2 | | | |
| Fragrance | 2.3 | 1.0 | | 0.5 |
| aluminum chloride | 5.0 | 10.0 | 5.0 | 4.5 |
| Glycine | 2.8 | 5.6 | 2.8 | 2.5 |
| propylene glycol | 5.0 | 35.0 | | 15.8 |
| water | 44.4 | 34.4 | 80.2 | 15.5 |
| FinSolve TN | 6.0 | | | |
| Performalene PL | 12.0 | | | |
| Dow 5225 formulation aid | | 7.0 | | 3.2 |
| Steareth-2 | | | 4.0 | |
| Steareth-21 | | | 2.0 | |
| PPG-15 Stearyl ether | | | 6 | |
| Isobutane | | | | 55.0 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aqueous antiperspirant composition comprising:
1) a metal salt with a M:X mole ratio from about 0.25 to 0.5, wherein X is an anion with a −1or −2 charge and M is a metal ion with a +2, +3 or +4 charge;
2) an amino acid wherein the M:amino acid mole ratio is from about 5 to about 0.33; and
3) wherein the pH of the composition is from about 2 to about 4.

2. The composition of claim 1, wherein the metal salt comprises a metal selected from the group consisting of aluminum, tin, zinc, gallium, iron, zirconium, titanium, magnesium, manganese, copper, silver, gold, calcium and strontium, and hafnium.

3. The composition of claim 1, wherein the metal salt comprises a metal selected from the group consisting of tin, zinc, titanium, magnesium, calcium and copper.

4. The composition of claim 1, wherein the metal salt comprises a metal selected from the group consisting of tin, zinc, and iron.

5. The composition of claim 1, wherein the metal salt is not aluminum.

6. The composition of claim 1, wherein the amino acid has an isoelectric point from about 5 to about 11.

7. The composition of claim 1, wherein the amino acid is selected from the group consisting of arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

8. The composition of claim 1, wherein the amino acid is selected from the group consisting of arginine, cysteine, and glycine.

9. The composition of claim 1, wherein the anion is selected from the group consisting of chloride, nitrate and sulfate.

10. The composition of claim 1, wherein the anion is a chloride ion.

11. The composition of claim 1, wherein the composition comprises at most about 10%, by weight of the composition, of the anhydrous metal salt.

12. An antiperspirant or deodorant composition comprising the composition of claim 1.
